(19) United States
(12) Patent Application Publication
(10) Pub. No.: US 2023/0321313 A1
(43) Pub. Date: Oct. 12, 2023

(54) Absorbent Materials from Dried Seaweed Flakes

(71) Applicant: ExxonMobil Chemical Patents Inc., Baytown, TX (US)

(72) Inventors: Glen E. Alliger, Houston, TX (US); Jason A. Mann, Houston, TX (US)

(21) Appl. No.: 18/189,524

(22) Filed: Mar. 24, 2023

Related U.S. Application Data
(60) Provisional application No. 63/330,054, filed on Apr. 12, 2022.

(51) Int. Cl. A61L 15/40; A61L 15/22; A61L 15/60

(52) U.S. Cl. CPC A61L 15/40; A61L 15/225; A61L 15/60

(57) ABSTRACT

A variety of methods and compositions are disclosed, including, in one embodiment, a method of making a superabsorbent material including: providing dried seaweed flakes comprising κ-carrageenan; hydrating the dried seaweed flakes to produced hydrated seaweed flakes; contacting the hydrated seaweed flakes with a non-aqueous solvent thereby producing the superabsorbent material; and drying the superabsorbent material.

### CROSS REFERENCE TO RELATED APPLICATIONS

This application claims the benefit of and priority to U.S. Provisional Application No. 63/330,054 filed Apr. 12, 2022, the disclosure of which is incorporated herein by reference

### FIELD

The present application relates to methods for producing superabsorbent materials from seaweed containing κ-carrageenan.

### BACKGROUND

Hygiene articles such as disposable diapers for infants and adults, incontinent pads, sanitary napkins, and pantiliners constitute major industries and serve important functions for different demographics of the population. In general, such hygiene articles are made from a skin-facing layer or an inner top sheet (also called a cover or front sheet) which is liquid-permeable to facilitate entry of the fluid exudate from the wearer into the hygiene article, a core of assonant material for absorbing liquid received through the top sheet, and an outer back sheet formed of a liquid impermeable plastic to eliminate leakage of fluid from the hygiene article. The back sheet may be vapor impermeable or vapor permeable. If vapor permeable, the product is said to be “breathable”.

Most hygiene articles such as diapers and sanitary napkin products use partially neutralized poly(acrylic acid) (PAA) that has been lightly crosslinked as the absorbent material. These materials are often referred to as Super-Absorbent Polymers (SAPs), because they are capable of absorbing many times their weight in water. SAPs are also used in agriculture to manage moisture and release nutrients. In spaces where PAA is so prevalent, “PAA” and “SAP” are often used interchangeably. The global market for PAA is extremely large given the number of hygiene articles used globally every year. While there has been some work to derive an SAP from natural polymer cellulose, extraction of the cellulose from the rest of the plant (lignin, hemicellulose) is cost prohibitive as compared to PAA, for example.

### SUMMARY

Disclosed herein is an example method of making a superabsorbent material comprising: providing dried seaweed flakes comprising κ-carrageenan; hydrating the dried seaweed flakes to produced hydrated seaweed flakes; contacting the hydrated seaweed flakes with a non-aqueous solvent thereby producing the superabsorbent material; and drying the superabsorbent material.

Further disclosed herein is an example superabsorbent material comprising: kappaphycus particles comprising crosslinked k-carrageenan, wherein about 0.5% or more of the k-carrageenan is crosslinked, wherein the kappaphycus particles comprise about 0.5% or less water by weight, wherein the kappaphycus particles have an average particle size in a range of about 0.01 mm to about 1 mm, and wherein the superabsorbent material has a property of absorbing about 5 times or more of its own weight when added to distilled water at about 20° C. and about 101.325 kPa.

Further disclosed herein is an example hygiene article comprising: a top sheet; an absorbent core comprising a superabsorbent material, wherein the superabsorbent material comprises kappaphycus particles comprising crosslinked k-carrageenan, wherein about 0.5% or more of the k-carrageenan is crosslinked, wherein the kappaphycus particles comprise about 0.5% or less water by weight, wherein the kappaphycus particles have an average particle size in a range of about 0.01 mm to about 1 mm, and wherein the superabsorbent material has a property of absorbing about 5 times or more of its own weight when added to distilled water at about 20° C. and about 101.325 kPa.; and a back sheet.

### BRIEF DESCRIPTION OF THE DRAWINGS

To assist those of ordinary skill in the relevant art in making and using the subject matter hereof, reference is made to the appended drawings, wherein:

FIG. 1 is an illustrative depiction of a process for making superabsorbent materials from dried seaweed flakes in accordance with certain embodiments of the present disclosure.

FIG. 2a is an illustrative depiction of a hygiene article including an absorbent core in a dry unexpanded state.

FIG. 2b is an illustrative depiction of a hygiene article including an absorbent core in an expanded state.

### DETAILED DESCRIPTION

Disclosed herein are superabsorbent materials and, more particularly, disclosed are superabsorbent materials derived from dried seaweed containing κ-carrageenan. Further disclosed herein are hygiene articles containing superabsorbent materials derived from dried seaweed containing κ-carrageenan. Seaweed can be readily grown in ocean farms at scale and is a renewable resource. Superabsorbent materials of the present disclosure provide an alternative source of superabsorbent polymer for hygiene articles which do not rely on hydrocarbon-derived materials. Superabsorbent polymers derived from seaweed are suitable as drop-in replacements for superabsorbent polymers such as PAA and cellulose based superabsorbent polymers. The term “superabsorbent material” as used herein is understood to mean a material which in its dry state (defined as having a total water content of 1% or less by weight), which when added to distilled water at 20° C. and 101.325 kPa will spontaneously absorb at least 5 times its own weight of the distilled water.

Superabsorbent materials of the present disclosure are derived from dried seaweed containing κ-carrageenan, a natural polysaccharide containing organosulfate and hydroxyl functional groups. The presence of the organosulfate and hydroxyl groups cause the κ-carrageenan to be hydrophilic and able to absorb water to form a thermo-reversible gel. A pure κ-carrageenan gel is readily soluble in water and will dissolve with additional water addition. In applications where the gel needs to be stable to keep water without dissolution, a crosslinking agent is used to modify the gel to form a hydrogel.

In some embodiments, a method of making a superabsorbent material from dried seaweed flakes containing κ-carrageenan includes sizing the dried seaweed flakes to produce sized seaweed flakes, hydrating sized the dried seaweed flakes to produce hydrated seaweed flakes, crosslinking the hydrated seaweed flakes to produce superabsorbent material, and dewatering the superabsorbent material to produce a dried superabsorbent material. The dried superabsorbent material can then be included in the production of a hygiene article as an absorbent core in the hygiene article. Dried seaweed flakes containing κ-carrageenan can be directly transformed to superabsorbent materials without the need to further purify or separate the κ-carrageenan from the bulk dried seaweed flake materials. Dried seaweed flakes can be used to produce the superabsorbent material “as is” without affecting the performance of the resulting superabsorbent material.

In some embodiments, the seaweed is red algae (Rhodophyta), and more particularly, seaweed from the genus kappaphycus. Any species of kappaphycus can be used in the superabsorbent materials of the present application, including Kappaphycus alvarezii, Kappaphycus cottonii, Kappaphycus inermis, Kappaphycus malesianus, Kappaphycus procrusteanus, Kappaphycus striatus, and combinations thereof.

A first step in producing superabsorbent materials from dried seaweed containing κ-carrageenan includes reducing the dried seaweed from a first average particle size to a second average particle size. Size reduction can be accomplished by any suitable means such as grinding, chopping, jet milling, ball milling, roller milling, or any other suitable method for reducing the average particle size of the dried seaweed to produce dried seaweed particles. The dried seaweed particles can have any average particle size after size reduction, such as an average particle at a point in a range of from 0.001 millimeters (“mm”) to 10 mm. Alternatively, the dried seaweed particles have an average particle size in a range of from 0.001 mm to 0.01 mm, from 0.01 mm to 0.1 mm, from 0.1 mm to 0.5 mm, from 0.5 mm to 0.1 mm, from 0.1 mm to 1 mm, from 1 mm to 10 mm, or any ranges therebetween. In some embodiments, the size reduced particles are further fractionated to a desired particle size range. Any suitable fractionation method can be used such as sieving, for example. While any fraction of the dried seaweed can be used, fractionating the particles into size range after size reduction allows for the fractions of dried seaweed particles to be selected for a particular application. Fractionating the dried seaweed particles has additional benefits such as increasing product consistency. In some embodiments, the dried seaweed particles can be fractionated such that the particles have a particle size in a range of from 0.001 mm to 0.01 mm, from 0.01 mm to 0.05 mm, from 0.05 to 0.1 mm, from 0.05 mm to 0.5 mm, from 0.5 mm to 1 mm, from 0.1 mm to 1 mm or any ranges therebetween.

A second step in producing superabsorbent materials from dried seaweed containing κ-carrageenan includes hydrating the dried seaweed particles after size reduction. In some embodiments, the hydrating can be performed with the entirety of the size reduced particles and/or a sized fraction of the size reduced dried seaweed particles. Hydrating the dried seaweed particles can be performed at any suitable conditions including at elevated temperature. In some embodiments, the hydrating is carried out at a temperature in a range of from 1° C. to 99° C., where relatively higher temperatures should allow for a shorter hydration time. Alternatively, hydrating is carried out at a temperature in a range of from 1° C. to 20° C., 20° C. to 30° C., 30° C. to 40° C., 40° C. to 50° C., 50° C. to 60° C., 60° C. to 70° C., 70° C. to 80° C., 80° C. to 90° C., 90° C. to 99° C., or any ranges therebetween. Hydration is carried out for any suitable length of time, for example, from 10 minutes to 24 hours. Alternatively, hydration is carried out from 10 minutes to 1 hour, 1 hour to 2 hours, 2 hours to 5 hours, 5 hours to 10 hours, 10 hours to 15 hours, 15 hours to 24 hours, or any ranges therebetween. It is not envisioned that there is an upper limit to hydration for the dried seaweed particles, however, the dried seaweed particles should be hydrated enough such that the crosslinking step can occur. Hydrating the dried seaweed particles can be carried out in any suitable vessel and with any source of water which does not contain chemical species which would detrimentally affect the resultant hydrated seaweed particles. In some embodiments, the dried seaweed particles can be hydrated at elevated temperatures for a period of time. For example, the dried seaweed flakes can be hydrated in water at 90° C. or greater for a period of 15 minutes or longer. The dried seaweed flakes can be hydrated in water at 50° C. or greater for a period of 15 minutes or longer.

A third optional step in producing superabsorbent materials from dried seaweed containing κ-carrageenan includes crosslinking the hydrated seaweed particles using a crosslinking agent thereby producing the superabsorbent material. Crosslinking can improve the mechanical properties of the superabsorbent material making the superabsorbent material more suitable for use in hygiene articles, for example. Any crosslinker capable of crosslinking κ-carrageenan may be used. In some embodiments, the crosslinking agent can include ionic crosslinkers such as, without limitation, calcium chloride and chemical crosslinkers such as, without limitation, polyaldehydes such as glutaraldehyde, adipic acid dihydrazide, 1,6-hexa-methylenediisocyanate or 1,6-hexanedibromide, NN-(3-dimethylaminopropyl)-N-ethyl carbodiimide, glutamic acid, citric acid mixed with sodium hyphophosphite, poly(carboxylic acid)s, and combinations thereof. The exact method of crosslinking is dependent upon the crosslinking agent chosen, such as in Formula 1 for example, where aldehyde groups from glutaraldehyde are reacted with hydroxyl groups in the κ-carrageenan which forms acetal bridges between adjacent κ-carrageenan. Any suitable amount of crosslinking agent can be used to crosslink the hydrated seaweed particles to produce the superabsorbent material. In some embodiments, the degree of crosslinking of the κ-carrageenan ranges from where 0.1% of the κ-carrageenan is crosslinked to where 50% κ-carrageenan is crosslinked. Alternatively, the κ-carrageenan is crosslinked from 0.1% to 0.5%, from 0.5% to 1%, from 1% to 5%, from 5% to 10%, from 10% to 25%, or from 25% to 50%. Degree of crosslinking can be determined by any suitable method such as those outlined in ASTM D2765 and ASTM F2214, for example.

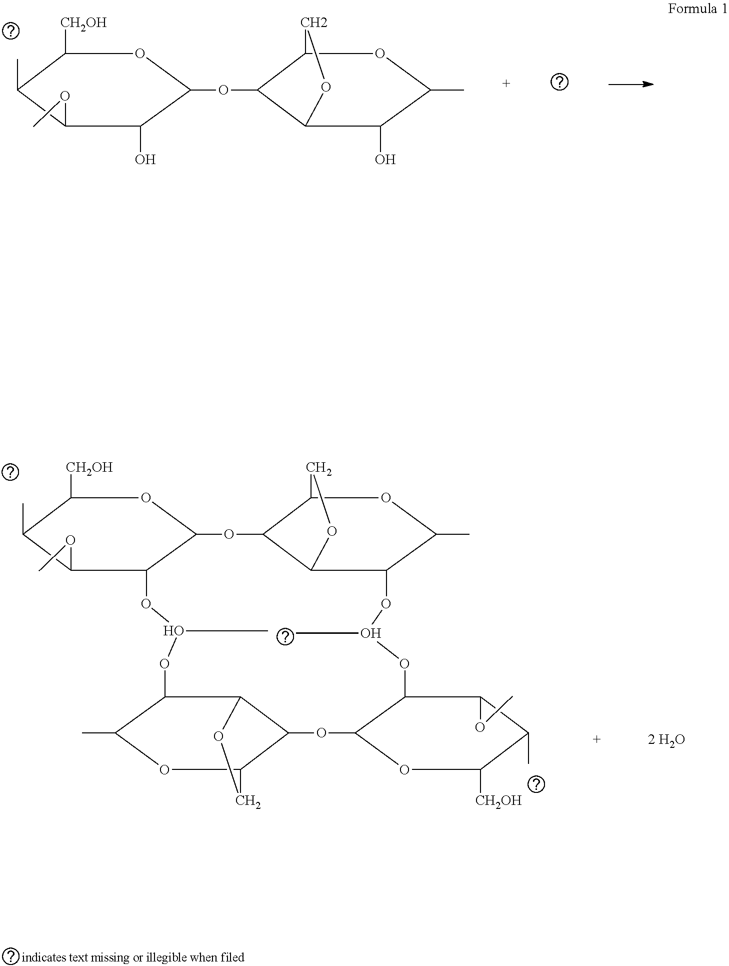

After crosslinking, a non-aqueous solvent is optionally added to the superabsorbent material to remove impurities from the superabsorbent material. In some embodiments the non-aqueous solvent is directly added to the aqueous solution containing the superabsorbent material. In further embodiments the superabsorbent material is separated from the aqueous solution and mixed with a non-aqueous solvent. In some embodiments where there is no crosslinker, the hydrated dried seaweed flakes are contacted with the non-aqueous solvent, either in an aqueous solution or separated from the aqueous solution and mixed with non-aqueous solvent to form the superabsorbent material. In some embodiments, the non-aqueous solvent is polar and/or non-polar. In some embodiments, the non-aqueous solvent includes alcohols such as methanol, ethanol, propanol, or hydrocarbon solvents such as C₅-C₁₆ hydrocarbons.

A fourth step in producing superabsorbent materials from dried seaweed containing κ-carrageenan includes drying the superabsorbent material to produce dried superabsorbent material. The superabsorbent material can be dried by any suitable method such as in an oven at a temperature suitable to dry the superabsorbent material to form a dried superabsorbent material. The dried superabsorbent material can be dried to any dryness including less than 1 wt. % water, less than 0.1 wt. % water, less than 0.01 wt. % water, or wherein the less than 0.001 wt. % water.

Another optional step in producing superabsorbent materials includes size reduction of superabsorbent materials particles after synthesis. Size reduction can be accomplished by any suitable means such as grinding, chopping, jet milling, ball milling, roller milling, or any other suitable method for reducing the average particle size of the superabsorbent materials. The superabsorbent materials can have any average particle size after size reduction, such as an average particle at a point in a range of from 0.001 mm to 10 mm. Alternatively, the superabsorbent materials have an average particle size in a range of from 0.001 mm to 0.01 mm, from 0.01 mm to 0.1 mm, from 0.1 mm to 0.5 mm, from 0.5 mm to 0.1 mm, from 0.1 mm to 1 mm, from 1 mm to 10 mm, or any ranges therebetween. In some embodiments, the size reduced superabsorbent materials are further fractionated to a desired particle size range. Any suitable fractionation method can be used such as sieving, for example.

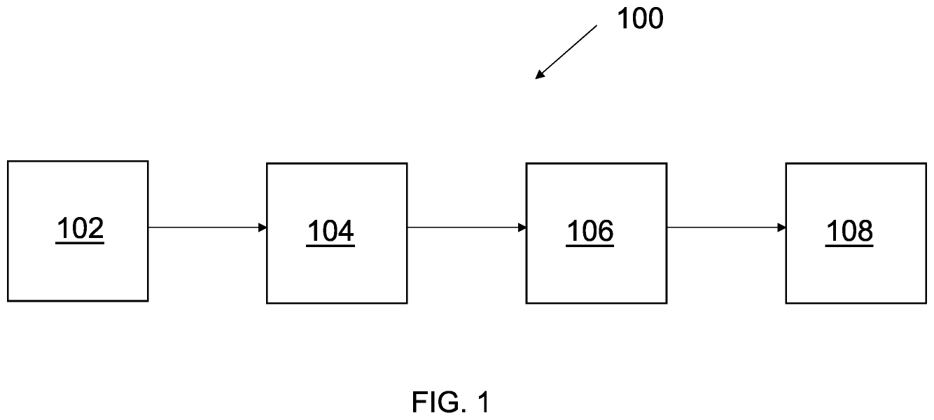

FIG. 1 illustrates a process 100 for producing superabsorbent materials from dried seaweed containing κ-carrageenan. Process 100 begins with block 102 where seaweed containing κ-carrageenan is provided and is reduced in size from a first average particle size to a second average particle size and size selected. In block 104 the dried seaweed particles from block 102 are hydrated to produce hydrated seaweed particles. In block 106 the hydrated seaweed particles from block 104 are crosslinked using a crosslinking agent to produce superabsorbent material. In block 108, the superabsorbent material from block 106 is dried to form a dried superabsorbent material. The dried superabsorbent material can be packaged or further processed to form an absorbent core suitable for use in a hygiene article.

Hygiene articles such as diapers for infants, adults, and animals, sanitary pads, sanitary napkins, tampons, and pantiliners, and generally include a top sheet, an absorbent core, and a back sheet. The top sheet is typically the layer of the hygiene article which is oriented towards and contacts the body of the wearer and is therefore the first layer to receive fluid. The top sheet is typically constructed from a single layer but alternatively includes more than one layer (e.g., a central top sheet layer and two overlapping lateral stripes). For example, a single non-woven material is used, in some embodiments, but the top sheet may be composed of several layers and treated to become hydrophilic so the liquid can pass through. The top sheet is normally permeable to liquids allowing liquids to pass through the top sheet without significantly retarding or obstructing the transmission of the fluid through the top sheet. The top sheet is prepared from any suitable materials. Some top sheet materials include, without limitation, nonwoven materials and perforated polyolefin films. An exemplary top sheet is a relatively hydrophobic 20 gsm (grams per square meter) spunbonded nonwoven web including fibers such as polypropylene and/or polyethylene polymers. In some embodiments, the top sheet is treated with a surfactant to enhance liquid penetration to the absorbent core. The top sheet can include a plurality of apertures to permit liquids deposited thereon to pass through to the core more quickly.

The hygiene articles further include an absorbent core disposed between the top sheet and the back sheet. The term absorbent core refers to a material or combination of materials suitable for absorbing, trapping, distributing, and/or storing fluids, for example, urine, blood, menses, and/or other exudates. The size and shape of the absorbent core may be such that the surface of the core in the horizontal plane is smaller than the surface of the top sheet. In some embodiments, the absorbent core is generally centered in the middle of the article and disposed away from the periphery of the article to provide improved flexibility along the edges of the article. The absorbent core may be fashioned into many shapes, for example, rounded, oval, rectangular, and square. However, flexibility and compatibility with various styles of undergarments may be better with cores having a curved shape such as an oval shape.

The absorbent core includes a superabsorbent material derived from dried seaweed containing κ-carrageenan as disclosed above. Dried superabsorbent material is formed into an absorbent core by any suitable methods. In some embodiments, the absorbent core further includes other suitable absorbent materials, including, but not limited to, comminuted wood pulp which is generally referred to as air felt, creped cellulose wadding; absorbent gelling materials including superabsorbent polymers such as hydrogel-forming polymeric gelling agents, chemically stiffened, modified, or cross-linked cellulose fibers, melt blown polymers including co-form, synthetic fibers including crimped polyester fibers, tissue including tissue wraps and tissue laminates, capillary channel fibers, absorbent foams, absorbent sponges, synthetic staple fibers, peat moss, foamed polyethylene or polypropylene compositions, nonwoven polyethylene or polypropylene materials, or any equivalent material; or combinations thereof. The absorbent core can include superabsorbent polymers (SAP) such as partially neutralized poly(acrylic acid) (PAA), normally distributed within a matrix of cellulosic fibers, for example, in order to reduce the thickness of the absorbent core. The absorbent core can be a monolayer or can be a laminate of two or more layers. For example, the core may comprise a fluid impermeable barrier layer on its back sheet-facing side to prevent fluids retained by the absorbent core from striking through the hygiene article.

The back sheet is positioned on the opposite side of the absorbent core to prevent discharges absorbed by the core from escaping the hygiene article. In some embodiments, the back sheet includes any suitable material which is generally flexible and impermeable to liquids. In some embodiments, the back sheet includes at least a film that is monolayer or two or more layers. The films may be cast or blown films, optionally, embossed and/or oriented or stretched, in either direction: Machine Direction (MD) or Transverse Direction (TD), either on-line or off-line. The films are optionally co-extruded. This back sheet also optionally contains fillers, pigment, various additives, and any combination thereof. The back sheet also optionally is a laminate with non-woven fabric. In some embodiments, the back sheet includes polyolefin materials such as polyethylene and/or polypropylene. Embodiments of this disclosure are directed to both regular hygiene articles and breathable hygiene articles. In breathable hygiene articles, back sheets which are permeable to vapor are utilized. These breathable back sheets provide a cooler garment and permit some drying of the article while being worn. In general, these breathable back sheets are intended to allow the passage of vapor while retarding the passage of liquid. In some embodiments, breathable back sheets are obtained by creating micro voids in one or more layers of the back sheet. Micro voids may be created by the incorporation of fillers, such as calcium carbonate (CaCO₃) or other suitable materials, into one or more layers of the back sheet followed by an orientation or stretching process. Other suitable materials include organic fillers such as polystyrene in polyethylene or water-swellable fillers such as silica or hydrogel. The back sheet has a garment-facing side and an opposite body-facing side. In some embodiments, the garment-facing side of the back sheet includes a non-adhesive area and an adhesive area.

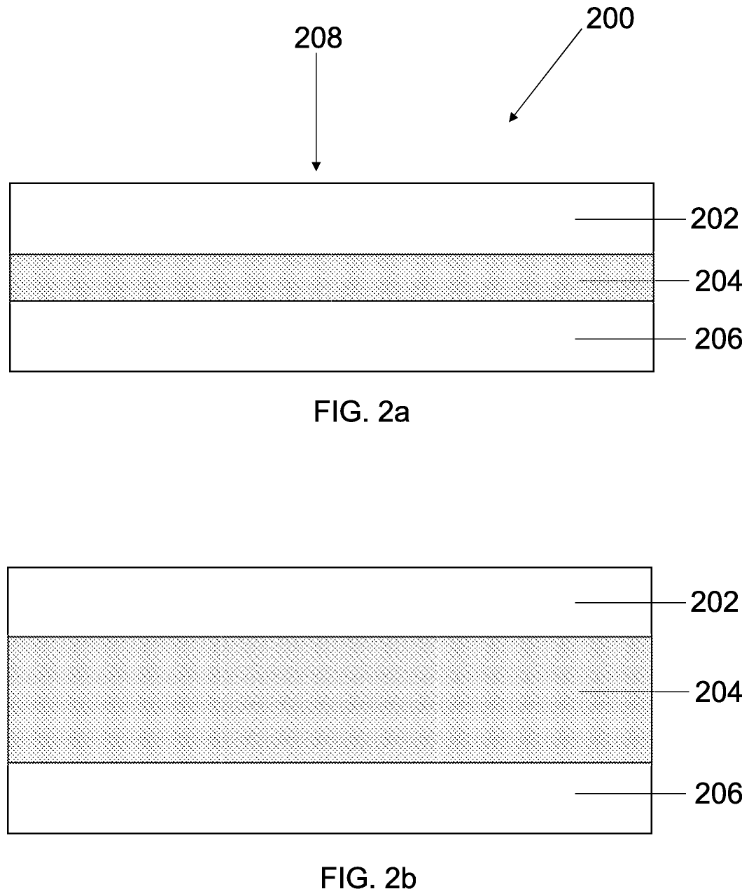

FIGS. 2a and 2b illustrate an embodiment of a hygiene article 200 including a top sheet 202, an absorbent core 204 which includes a superabsorbent material derived from dried seaweed containing κ-carrageenan as disclosed above, and a back sheet 206. In FIG. 2a, the absorbent core 204 is shown in a dry, unexpanded state. Top sheet 202 is permeable to liquids allowing liquids to flow through the top sheet 202 into absorbent core 204 as indicated by arrow 208. In FIG. 2b, absorbent core 204 is shown in an expanded state after liquid has contacted the absorbent core and the superabsorbent material within the absorbent core 204 has absorbed the liquid.

### Additional Embodiments

Accordingly, the present disclosure may provide methods for producing superabsorbent materials from seaweed containing κ-carrageenan.
Embodiment 1. A method of making a superabsorbent material comprising: providing dried seaweed flakes comprising κ-carrageenan; hydrating the dried seaweed flakes to produced hydrated seaweed flakes; contacting the hydrated seaweed flakes with a non-aqueous solvent thereby producing the superabsorbent material; and drying the superabsorbent material.
Embodiment 2. The method of embodiment 1 further comprising mixing the hydrated seaweed flakes with a crosslinking agent and crosslinking at least a portion of the κ-carrageenan.
Embodiment 3. The method of any of embodiments 1-2 wherein the crosslinking agent comprises an ionic crosslinking agent.
Embodiment 4. The method of any of embodiments 1-3 wherein the crosslinking agent comprises calcium chloride.
Embodiment 5. The method of any of embodiments 1-4 wherein the crosslinking agent comprises a chemical crosslinker selected from the group consisting of, polyaldehydes, adipic acid dihydrazide, 1,6-hexa-methylenediisocyanate or 1,6-hexanedibromide, NN-(3-dimethylaminopropyl)-N-ethyl carbodiimide, glutamic acid, citric acid mixed with sodium hyphophosphite, poly(carboxylic acid)s, and combinations thereof
Embodiment 6. The method of any of embodiments 1-5 further comprising reducing an average particle size of the dried seaweed flakes to produce reduced size dried seaweed flakes, prior to hydrating the dried seaweed flakes.
Embodiment 7. The method of any of embodiments 1-6 further comprising separating a fraction of dried seaweed flakes with a particle size of about 0.01 mm to about 0.1 mm from the dried seaweed flakes, wherein the fraction of dried seaweed flakes are hydrated to produce the hydrated seaweed flakes.
Embodiment 8. The method of any of embodiments 1-7 wherein the dried seaweed flakes comprise a species of kappaphycus selected from the group consisting of Kappaphycus alvarezii, Kappaphycus cottonii, Kappaphycus inermis, Kappaphycus malesianus, Kappaphycus procrusteanus, Kappaphycus striatus, and combinations thereof
Embodiment 9. The method of any of embodiments 1-8 wherein the non-aqueous solvent comprises an alcohol or a hydrocarbon.
Embodiment 10. The method of any of embodiments 1-10 further comprising reducing an average particle size of the superabsorbent material to an average particle size of from about 0.001 mm to about 0.1 mm.
Embodiment 11. A superabsorbent material comprising: kappaphycus particles comprising crosslinked k-carrageenan, wherein about 0.5% or more of the k-carrageenan is crosslinked, wherein the kappaphycus particles comprise about 0.5% or less water by weight, wherein the kappaphycus particles have an average particle size in a range of about 0.01 mm to about 1 mm, and wherein the superabsorbent material has a property of absorbing about 5 times or more of its own weight when added to distilled water at about 20° C. and about 101.325 kPa.
Embodiment 12. The superabsorbent material of embodiment 11 wherein the crosslinked k-carrageenan is crosslinked with a chemical crosslinker selected from the group consisting of, polyaldehydes, adipic acid dihydrazide, 1,6-hexa-methylenediisocyanate or 1,6-hexanedibromide, NN-(3-dimethylaminopropyl)-N-ethyl carbodiimide, glutamic acid, citric acid mixed with sodium hyphophosphite, poly(carboxylic acid)s, calcium chloride, and combinations thereof
Embodiment 13. A hygiene article comprising: a top sheet; an absorbent core comprising a superabsorbent material, wherein the superabsorbent material comprises kappaphycus particles comprising crosslinked k-carrageenan, wherein about 0.5% or more of the k-carrageenan is crosslinked, wherein the kappaphycus particles comprise about 0.5% or less water by weight, wherein the kappaphycus particles have an average particle size in a range of about 0.01 mm to about 1 mm, and wherein the superabsorbent material has a property of absorbing about 5 times or more of its own weight when added to distilled water at about 20° C. and about 101.325 kPa.; and a back sheet.
Embodiment 14. The hygiene article of embodiment 13 wherein the hygiene article comprises an adult diaper, an infant diaper, a sanitary pad, a sanitary napkin, or a pantiliner.
Embodiment 15. The hygiene article of any of embodiments 13-14 wherein the top sheet is permeable to liquids.
Embodiment 16. The hygiene article of any of embodiments 13-15 wherein the kappaphycus particles comprise a species of kappaphycus selected from the group consisting of Kappaphycus alvarezii, Kappaphycus cottonii, Kappaphycus inermis, Kappaphycus malesianus, Kappaphycus procrusteanus, Kappaphycus striatus, and combinations thereof
Embodiment 17. The hygiene article of any of embodiments 13-16 wherein the crosslinked k-carrageenan is crosslinked with a crosslinking agent comprising calcium chloride.
Embodiment 18. The hygiene article of any of embodiments 13-17 wherein the crosslinked k-carrageenan is crosslinked with a chemical crosslinker selected from the group consisting of, polyaldehydes, adipic acid dihydrazide, 1,6-hexa-methylenediisocyanate or 1,6-hexanedibromide, NN-(3-dimethylaminopropyl)-N-ethyl carbodiimide, glutamic acid, citric acid mixed with sodium hyphophosphite, and combinations thereof
Embodiment 19. The hygiene article of any of embodiments 13-18 wherein the absorbent core further comprises partially neutralized poly(acrylic acid).
Embodiment 20. The hygiene article of any of embodiments 13-19 wherein the back sheet comprises a breathable back sheet.

To facilitate a better understanding of the present invention, the following examples of certain aspects of some embodiments are given. In no way should the following examples be read to limit, or define, the entire scope of the disclosure.

### Example 1

### General Methodology

In these Examples, absorbent gels were prepared using dried seaweed flakes (DSF). The DSF is comprised of finely chopped, dried seaweed, and can be used directly in the crosslinking process, without further purification of the carrageenan from the material. Briefly, the process for making SAPs from DSF involves sieving, hydrating, crosslinking, and dewatering. The DSF used in each example was made from dried kappaphycus spp., and contains κ-carrageenan, a natural sulfated polysaccharide.

### Gel Synthesis

In this Example, 199.3 g of DSF was introduced to a food processor and blended on high for 10 minutes in 1-minute intervals. This led to a powder that was then sieved using consecutive sieve of progressively finer meshes. The different fractions were then collected individually on a large piece of paper, weighed, and stored in plastic containers. The weight of each mesh range DSF collected is shown in Table 1.

TABLE 1

| MESH | WEIGHT (g) |
|---|---|
| 8-14 | 19.866 |
| 14-18 | 54.273 |
| 18-25 | 46.468 |
| 25-35 | 34.157 |
| 35-45 | 18.839 |
| 45-120 | 16.43 |
| 120-170 | 1.259 |
| 170+ | 1.597 |

A series of gels were prepared according to Table 2. A measured mass 25-35 mesh DSF was introduced into a 100 mL round bottom flask. A stir bar and approximately 50 mL of deionized water was added to the flask. The flask was immersed in a 90° C. oil bath and stirred at 400 rpm. The flask was capped after five minutes, and the mixture was allowed to stir for 2 hours. After the time period had elapsed, the flask was opened and crosslinker was optionally added as shown in Table 2. The mixture was again allowed to stir at 90° C. for 15 minutes. Heat was removed, and the mixture was allowed to cool to ambient over 24 hours. After 24 hours, the mixture was transferred into a beaker and methanol was added until the total volume was 1 L. This mixture was allowed to equilibrate for 24 hours. The mixture was filtered, and residue was collected and washed with methanol. The solid thus obtained was dried to constant weight in a vacuum oven at 60° C. Additionally, gel 19* was dewatered with 500 mL methanol and gel 21** was crosslinked for a period of 2 hours.

TABLE 2

| | | | Crosslinker |
|---|---|---|---|
| Gel # | Mass of DSF (g) | Crosslinker(s) | mass (mg) |
| 1 | 1.0252 | CaCl2 | 50 |
| 2 | 1.0564 | CaCl2 | 54 |
| 3 | 1.0217 | Citric Acid, NaH2PO2 | 25, 7.5 |
| 4 | 1.0518 | CaCl2 | 52.3 |
| 5 | 1.0062 | CaCl2 | 51.3 |
| 6 | 1.0092 | Citric Acid, NaH2PO2 | 23.6, 3.6 |
| 7 | 1.0073 | Glutaraldehyde | 50 |
| 8 | 1.0036 | Glutaraldehyde | 50 |
| 9 | 1.0143 | Glutaraldehyde | 50 |
| 10 | 1.0076 | Citric Acid, NaH2PO2 | 23.5, 3.2 |
| 11 | 1.0025 | None | — |
| 12 | 1.0175 | None | — |
| 13 | 1.0065 | None | — |
| 14 | 1.000 (120-170 mesh) | CaCl2 | 10 |
| 15 | 1.045 | CaCl2 | 10 |
| 16 | 1.016 | CaCl2 | 50 |
| 17 | 1.014 | citric acid | 10 |
| 18 | 1.002 | glyceraldehyde | 10 |
| 19* | 1 | CaCl2 | 10 |
| 20 | 1 | glutaral dehyde | 50 |
| 21** | 1 | glutaral dehyde | 10 |
| 22 | 1 | DL-glutamic acid | 10 |

Two control gels were prepared from purified κ-carrageenan as shown in Table 2 using the previously discussed methodology.

TABLE 3

| Gel # | Mass of κ-C (g) | Crosslinker(s) | Crosslinker mass (mg) |
|---|---|---|---|
| 23 | 1.015 | CaCl2 | 10 |
| 24 | 0.996 | none | — |

### Gel Testing

Immersion swelling testing was performed on the synthesized gels using two different methods. The swelling testing for method “A” were performed in the following manner: a measured amount of dewatered, dried gel was added to a teabag of known weight. The prepared teabag was immersed in a 0.9% saline solution in a jar for a period of time. Simultaneously, a second, empty teabag was immersed in a second jar for the same amount of time. After a period of time had elapsed, the teabags were removed from their respective jars, and patted dry with filter paper. Both teabags were weighed. The percent swelling was determined by Equation 1:

$$\frac{{M}_{SAP - immersed} - {M}_{empty}}{{M}_{SAP - initial}} * 100 \qquad Equation 1$$

where M_{SAP-immersed} is the mass of the teabag containing the SAP after immersion, M_{empty} is the mass of the empty teabag after immersion, and M_{SAP-init} is the mass of the DSF SAP prior to immersion.

A second immersion swelling test method “B” was performed in the following manner: a measured amount of dewatered, dried gel was added to a teabag of known weight. The prepared teabag was immersed in a 0.9% saline solution in a jar for a period of time. The teabag was removed from the jar and patted dry with a piece of filter paper. The percent swelling was determined by Equation 2.

$$\frac{{M}_{SAP - immersed} - {M}_{empty}}{{M}_{SAP - initial}} * 100 \qquad Equation 2$$

where M_{SAP-immersed} is the mass of the teabag containing the SAP after immersion, M_{empty} is the mass of the empty teabag prior to immersion, and M_{SAP-init} is the mass of the DSF SAP prior to immersion. The results of the swelling tests are shown in Table 4.

TABLE 4

| Gel # | Mass of SAP (g) | Time elapsed (h) | % Swelling | Method |
|---|---|---|---|---|
| 1 | 0.1057 | 1.22 | 525 | B |
| 2 | 0.1046 | 1.27 | 749 | B |
| 3 | 0.1207 | 1.38 | 1358 | B |
| 4 | 0.1005 | 1.22 | 2234 | B |
| 5 | 0.1162 | 1.17 | 543 | B |
| 6 | 0.1263 | 1.12 | 1938 | B |
| 7 | 0.1292 | 1.07 | 2122 | B |
| 8 | 0.1186 | 1.02 | 2269 | B |
| 9 | 0.1194 | 1 | 1563 | B |
| 10 | 0.1164 | 1.23 | 1625 | B |
| 11 | 0.1078 | 1.2 | 1309 | B |
| 12 | 0.0932 | 0.92 | 2270 | B |
| 13 | 0.0904 | 1.03 | 2141 | B |
| 14 | 0.1668 | 1 | 535 | A |
| 15 | 0.1165 | 1 | 1136 | A |
| 16 | 0.1005 | 1 | 1013 | A |
| 17 | 0.1098 | 1 | 441 | A |
| 18 | 0.1105 | 1 | 950 | A |
| 19 | 0.1161 | 1 | 404 | A |
| 20 | 0.1037 | 1 | 452 | A |
| 21 | 0.1404 | 1 | 848 | A |
| 23 | 0.1094 | 1 | 1763 | A |
| 24 | 0.1046 | 1 | 498 | A |

For comparative purposes, partially hydrolyzed poly(acrylic acid) was added to teabags using the method described above. Swelling tests were performed and the results of the swelling tests are shown in Table 5.

TABLE 5

| Gel # | Mass of SAP (g) | Time elapsed (h) | % Swelling | Method |
|---|---|---|---|---|
| C1 | 0.1138 | 1.25 | 4007% | B |
| C2 | 0.0956 | 1.33 | 2077% | B |
| C3 | 0.097 | 1.25 | 4809% | B |
| C4 | 0.0928 | 1.33 | 4385% | B |

It was observed that the crosslinked κ-carrageenan containing gels swelled similarly to the non-crosslinked κ-carrageenan containing gels.

### Example 2

In this example, an alternative synthesis method for producing a superabsorbent material is presented. The superabsorbent material was prepared using the same dried seaweed flakes (DSF) as in Example 1. DSF was ground and sieved to isolate particles with particle sizes of 0.707 mm to 0.5 mm. A 225-gram sample of the sieved DSF was added to a 4-liter beaker with 3.5 liters of deionized water. The mixture was stirred for 3.5 hours at 90° C. with the top loosely covered to hydrate the dried seaweed flakes.

Three samples of hydrated seaweed flakes were prepared. In two of the three samples, 5 wt. % crosslinker was added 15 minutes before the end of this the first heating period. The samples were as follows: A: no crosslinking agent used, B: 11.3 g of glutaraldehyde in 25% aqueous solution as crosslinking agent; C: 11.51 g of CaCl₂ as crosslinking agent. For each of the samples, the temperature was lowered to 60° C. and the mixture left to stir overnight with the top of the vessel uncovered.

The next morning, 2 liters of methanol was added to each sample and the mixture was stirred by hand for 10 minutes. This mixture was subsequently left to sit for 24 hours. Each mixture was then poured through two layers of cheesecloth, isolating the gelled solid. This solid was mixed with 2 liters of methanol, stirred by hand for 5 minutes, and then once again filtered with cheesecloth. The solid was left in the cheesecloth for several hours to allow for additional draining. When the drainage had come to a halt, the solid was placed in a vacuum oven and dried at 70° C. for 5 days. The material was removed once per day for hand mixing and to break up any large conglomerates. After this treatment, the gel was reduced to hard dark brown pellets of various sizes. The resultant yields for each sample were as follows: Sample A: 169 g; Sample B: 183 g; Sample C: 200 g.

Three immersion swell tests were performed for each sample. The results of the immersion test are shown in Table 6.

TABLE 6

| Sample | Sample | Time (h) | End Weight | Water Weight | % Increase |
|---|---|---|---|---|---|
| A1 | 0.103 | 1.10 | 2.3101 | 1.7631 | 1712% |
| A2 | 0.1153 | 1.03 | 1.931 | 1.3801 | 1197% |
| A3 | 0.1388 | 1.00 | 2.3305 | 1.7704 | 1276% |
| B1 | 0.1347 | 1.05 | 11.825 | 2.0682 | 1535% |
| B2 | 0.1124 | 1.05 | 6.58 | 2.0528 | 1826% |
| B3 | 0.0935 | 1.02 | 5.7733 | 1.2263 | 1312% |
| C1 | 0.1184 | 1.08 | 7.305 | 1.0861 | 917% |
| C2 | 0.1135 | 1.07 | 5.5525 | 1.021 | 900% |
| C3 | 0.1398 | 1.03 | 5.6853 | 0.9437 | 675% |

### Example 3

In this example, unmodified dried seaweed flakes, superabsorbent material produced from hydrated seaweed flakes as gels 11, 12, and 13 from Example 1, and powdered superabsorbent material produced from gels 11, 12, and 13 were swell tested. The results of the swell testing are show in Table 7. It was observed that gels 11, 12, and 13 which are not crosslinked performed better than unmodified DSF. Gels 11, 12, and 13 were prepared by hydrating at elevated temperature which increased the amount of swelling over the unmodified DSF. The powdered gels were observed to have the most swelling of all the tested gels.

TABLE 7

| Gel # | % Swelling |
|---|---|
| Gel 11 | 1309% |
| Gel 12 | 2270% |
| Gel 13 | 2141% |
| Powder gel 1 | 2158% |
| Powder gel 2 | 2206% |
| Powder gel 3 | 2214% |
| Powder gel 4 | 2195% |
| Unmodified DSF 1 | 1246% |
| Unmodified DSF 2 | 1538% |
| Unmodified DSF 3 | 1424% |
| Unmodified DSF 4 | 1201% |

While the disclosure has been described with respect to a number of embodiments and examples, those skilled in the art, having benefit of this disclosure, will appreciate that other embodiments can be devised which do not depart from the scope and spirit of the disclosure as disclosed herein. Although individual embodiments are discussed, the present disclosure covers all combinations of all those embodiments.

While compositions, methods, and processes are described herein in terms of “comprising,” “containing,” “having,” or “including” various components or steps, the compositions and methods can also “consist essentially of” or “consist of” the various components and steps. The phrases, unless otherwise specified, “consists essentially of” and “consisting essentially of” do not exclude the presence of other steps, elements, or materials, whether or not, specifically mentioned in this specification, so long as such steps, elements, or materials, do not affect the basic and novel characteristics of the disclosure, additionally, they do not exclude impurities and variances normally associated with the elements and materials used.

All numerical values within the detailed description are modified by “about” the indicated value, and take into account experimental error and variations that would be expected by a person having ordinary skill in the art.

Many alterations, modifications, and variations will be apparent to those skilled in the art in light of the foregoing description without departing from the spirit or scope of the present disclosure and that when numerical lower limits and numerical upper limits are listed herein, ranges from any lower limit to any upper limit are contemplated.

1. A method of making a superabsorbent material comprising:
hydrating dried seaweed flakes, which include κ-carrageenan, to produce hydrated seaweed flakes;
contacting the hydrated seaweed flakes with a non-aqueous solvent thereby producing the superabsorbent material; and
drying the superabsorbent material.

2. The method of claim 1, further comprising mixing the hydrated seaweed flakes with a crosslinking agent and crosslinking at least a portion of the κ-carrageenan.

3. The method of claim 2, wherein the crosslinking agent comprises an ionic crosslinking agent.

4. The method of claim 2, wherein the crosslinking agent comprises calcium chloride.

5. The method of claim 2, wherein the crosslinking agent comprises a chemical crosslinker selected from the group consisting of, polyaldehydes, adipic acid dihydrazide, 1,6-hexa-methylenediisocyanate or 1,6-hexanedibromide, NN-(3-dimethylaminopropyl)-N-ethyl carbodiimide, glutamic acid, citric acid mixed with sodium hyphophosphite, poly(carboxylic acid)s, and combinations thereof.

6. The method of claim 1, further comprising reducing an average particle size of the dried seaweed flakes to produce reduced size dried seaweed flakes, prior to hydrating the dried seaweed flakes.

7. The method of claim 6, further comprising separating a fraction of dried seaweed flakes with a particle size of about 0.01 mm to about 0.1 mm from the dried seaweed flakes, wherein the fraction of dried seaweed flakes are hydrated to produce the hydrated seaweed flakes.

8. The method of claim 1, wherein the dried seaweed flakes comprise a species of kappaphycus selected from the group consisting of Kappaphycus alvarezii, Kappaphycus cottonii, Kappaphycus inermis, Kappaphycus malesianus, Kappaphycus procrusteanus, Kappaphycus striatus, and combinations thereof.

9. The method of claim 1, wherein the non-aqueous solvent comprises an alcohol or a hydrocarbon.

10. The method of claim 1, further comprising reducing an average particle size of the superabsorbent material to an average particle size of from about 0.001 mm to about 0.1 mm.

11. An article, comprising:
kappaphycus particles comprising crosslinked k-carrageenan,
wherein about 0.5% or more of the k-carrageenan is crosslinked, wherein the kappaphycus particles comprise about 0.5% or less water by weight, wherein the kappaphycus particles have an average particle size in a range of about 0.01 mm to about 1 mm, and wherein the superabsorbent material has a property of absorbing about 5 times or more of its own weight when added to distilled water at about 20° C. and about 101.325 kPa.

12. The article of claim 11, wherein the crosslinked k-carrageenan is crosslinked with a chemical crosslinker selected from the group consisting of, polyaldehydes, adipic acid dihydrazide, 1,6-hexa-methylenediisocyanate or 1,6-hexanedibromide, NN-(3-dimethylaminopropyl)-N-ethyl carbodiimide, glutamic acid, citric acid mixed with sodium hyphophosphite, poly(carboxylic acid)s, calcium chloride, and combinations thereof.

13. A hygiene article, comprising:
a top sheet;
an absorbent core comprising the article of claim 11; and
a back sheet.

14. The hygiene article of claim 13, wherein the hygiene article comprises an adult diaper, an infant diaper, a sanitary pad, a sanitary napkin, or a pantiliner.

15. The hygiene article of claim 13, wherein the top sheet is permeable to liquids.

16. The hygiene article of claim 13, wherein the kappaphycus particles comprise a species of kappaphycus selected from the group consisting of Kappaphycus alvarezii, Kappaphycus cottonii, Kappaphycus inermis, Kappaphycus malesianus, Kappaphycus procrusteanus, Kappaphycus striatus, and combinations thereof.

17. The hygiene article of claim 13, wherein the crosslinked k-carrageenan is crosslinked with a crosslinking agent comprising calcium chloride.

18. The hygiene article of claim 13, wherein the crosslinked k-carrageenan is crosslinked with a chemical crosslinker selected from the group consisting of, polyaldehydes, adipic acid dihydrazide, 1,6-hexa-methylenediisocyanate or 1,6-hexanedibromide, NN-(3-dimethylaminopropyl)-N-ethyl carbodiimide, glutamic acid, citric acid mixed with sodium hyphophosphite, and combinations thereof.

19. The hygiene article of claim 13, wherein the absorbent core further comprises partially neutralized poly(acrylic acid).

20. The hygiene article of claim 13, wherein the back sheet comprises a breathable back sheet.
